(19) United States
(12) Patent Application Publication
(10) Pub. No.: US 2008/0312509 A1
(43) Pub. Date: Dec. 18, 2008

(54) Surgical Clamp For Cylindrical Stock

(75) Inventors: Steven Karl JACOBSON, St. Paul, MN (US); Todd M. BJORK, River Falls, WI (US)

Correspondence Address: SHEWCHUK IP SERVICES, 3356 SHERMAN CT. STE. 102, EAGAN, MN, 55121, US

(73) Assignee: LEVAHN INTELLECTUAL PROPERTY HOLDING COMPANY, LLC, St. Paul, MN (US)

(21) Appl. No.: 11/762,035

(22) Filed: Jun. 12, 2007

(51) Int. Cl. A61B 17/00

(52) U.S. Cl. 600/230; 248/689

(57) ABSTRACT

A U-type clamp has two arms and a base and tightens around cylindrical stock. The surface of the clamp that makes contact with the cylindrical stock is not itself entirely cylindrical in its loosened state. The preferred embodiment involves a first cylindrical profile extending around about ¾ of the contact surface which is intended to match the curvature of the post. The final ¼ or so of contact surface has a larger radius of curvature than the first cylindrical profile. The second cylindrical profile may also be offset relative to the first cylindrical profile.

### CROSS-REFERENCE TO RELATED APPLICATION(S)

None.

### BACKGROUND OF THE INVENTION

The present invention relates generally to the field of surgical tools, and particularly to the design and manufacture of surgical retractor systems, including clamps for use in mounting surgical retractors with respect to an operating table. More particularly, the present invention relates to a clamp that is tightened around a post or bar which is cylindrical in shape.

Surgical retractor systems are used during surgery to bias and hold tissue in a desired position. Tissue such as skin, muscle, fatty tissue and interior organs needs to be held retracted to the side so the surgeon can obtain better access to the surgical arena of primary interest. The retractor assembly may, for instance, include a ring or support frame which is rigidly supported from the patient's bed above and around the surgical incision location, with a number of clamps and retractor blades to hold back tissue proximate to the surgical incision. Retractor clamps are also commonly used to mount the retractor support apparatus with respect to an operating table, support post and/or part of the bed frame.

In one style of clamping member, the clamping member is fabricated from a unitary structure that is generally in the shape of the letter “U”. The object to which the clamp is affixed can be cylindrical bar stock having a circular cross-section generally matching the diameter of the base of the “U” shape. The cylindrical post or bar clamped is placed between the legs of the U-shaped structure proximate the base of the U-shaped structure. Movement of the legs of the U-shaped structure towards each other causes the clamp to tighten onto the post or bar.

Such U-shaped clamps have long been in use in the surgical retractor field. Generally, the inner diameter of the U-shape defines a cylindrical contact surface with a loosened diameter slightly larger than the outer diameter of the cylindrical stock.

Many such U-shaped clamps are tightened with a threaded bolt between the legs of the U-shape. The threaded bolt gives a large range of adjustment tightening the legs of the U-shape together.

In more advanced designs, the bolt may be tightened with a cam member, which typically has a more limited range of adjustment than screw tightening. In particular, the handle for a cam tightened bolt is often desired to have a defined tightened position in a relatively small range after a relatively longer throw. For instance, the throw of the cam handle may be through about 170°, with desired tightening occurring at 170±10°. If the diameter of the cylindrical stock is out of round or out of tolerance, it may affect the tightening throw of the cam handle, such that the handle may fall short or may travel through the desired tightened handle position. For example, if the diameter of the cylindrical stock is slightly smaller than expected, the cam handle may not tighten until a position of about 200°, which may place the handle in an awkward position for grasping when loosening is desired. Such inconsistencies make the clamp difficult to use in certain locations or in certain situations.

Surgical retractor systems must be robust and strong, as even a slight possibility of failure during use is not tolerated. Surgical retractor assemblies should be readily reusable, including sterilizable, for use in multiple surgeries. Surgical retractor systems should maintain a relatively low cost. Surgical retractor parts should allow retrofitting with all or a portion of previous systems that may already be present in the field. Improvements in surgical retractor clamps and systems can be made in keeping with these goals.

### BRIEF SUMMARY OF THE INVENTION

The present invention is a U-type clamp for tightening around cylindrical stock. However, the surface of the clamp that makes contact with the cylindrical stock is not itself entirely cylindrical as formed. The preferred embodiment involves a larger circumferential portion of a first cylindrical profile coupled with a smaller circumferential portion of a second cylindrical profile. The second cylindrical profile may be offset relative to the first cylindrical profile, and may have a slightly larger radius of curvature than the first cylindrical profile. The two profiles may be connected with a flat or a curve so as to avoid any inflection points in the contact profile.

### BRIEF DESCRIPTION OF THE DRAWINGS

FIG. 1 is a perspective view of a surgical clamp assembly utilizing two clamps in accordance with the present invention.

FIG. 2 is an end view in partial cross section of the surgical clamp assembly of FIG. 1.

FIG. 3 is a side view in partial cross section of the surgical clamp assembly of FIGS. 1 and 2.

FIG. 4 is a perspective view of one of the clamps in the clamp assembly of FIGS. 1-3.

FIG. 5 is a top view of the clamp of FIG. 4.

FIG. 6 is an enlarged side view of the clamp of FIGS. 4 and 5.

FIG. 7 is a cross-sectional side view of the clamp of FIGS. 4-6, taken along lines 7-7 in FIG. 5.

FIG. 8 is a cross-sectional side view showing the clamp portion of the clamp assembly in loosened position around cylindrical stock.

FIG. 9 is a cross-sectional side view showing the clamp portion of FIG. 8 in tightened position around cylindrical stock.

While the above-identified drawing figures set forth one or more preferred embodiments, other embodiments of the present invention are also contemplated, some of which are noted in the discussion. In all cases, this disclosure presents the illustrated embodiments of the present invention by way of representation and not limitation. Numerous other minor modifications and embodiments can be devised by those skilled in the art which fall within the scope and spirit of the principles of this invention.

### DETAILED DESCRIPTION

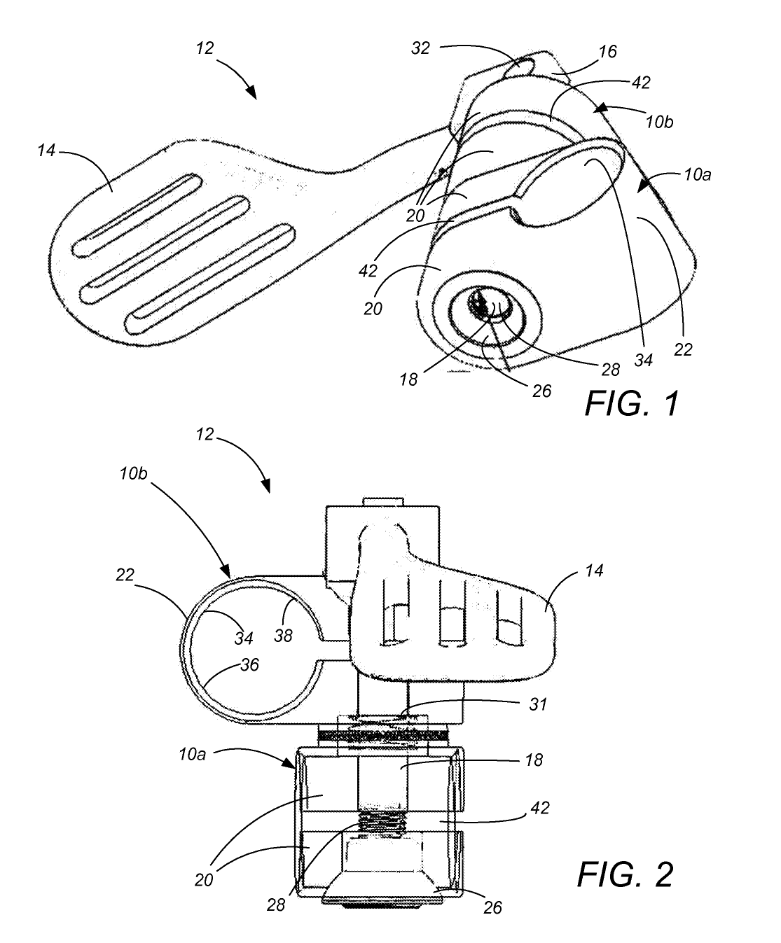

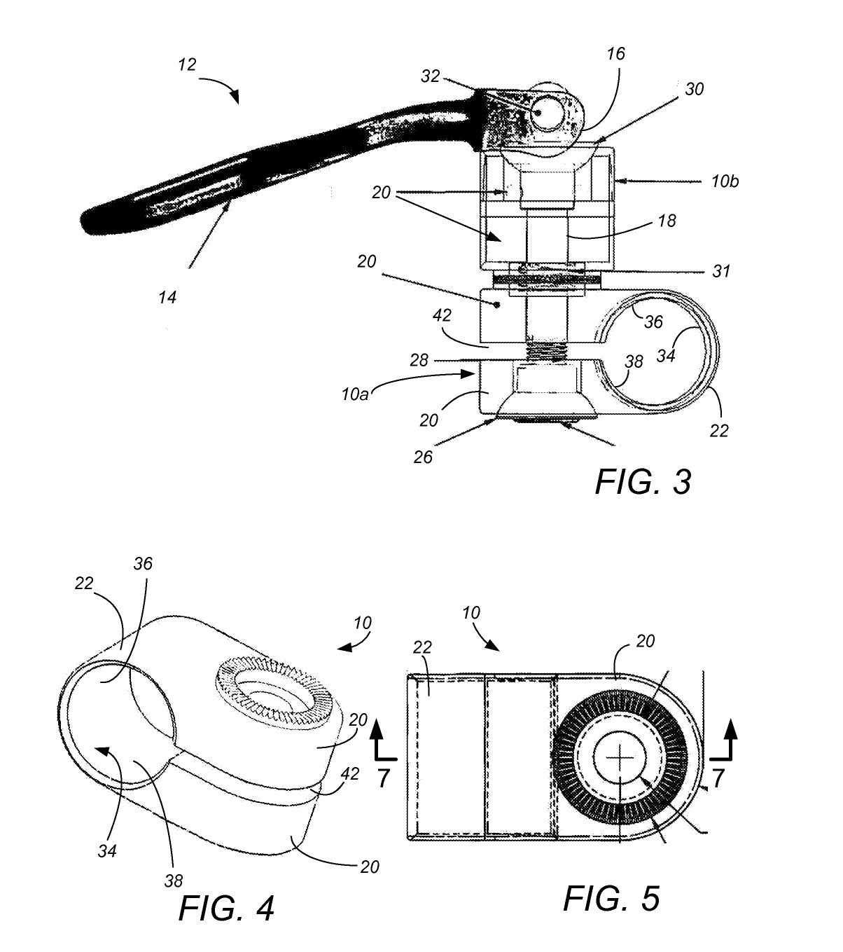

The present invention involves a U-type surgical clamp 10 which can be used in a surgical clamp assembly 12 as shown in FIGS. 1-3. The surgical clamp 10 can also be used in a variety of other locations, including any of the uses suggested in U.S. Pat. Nos. 4,718,151, 4,949,707, 5,020,195, 5,025,780, 5,888,197, 5,897,087, 6,017,306, and 6,264,396, all incorporated by reference. The clamp assembly 12 generally includes a first, outside clamp 10a, and second, inside clamp 10b, a handle 14 with an integrally formed cam 16, and a clamp bolt 18.

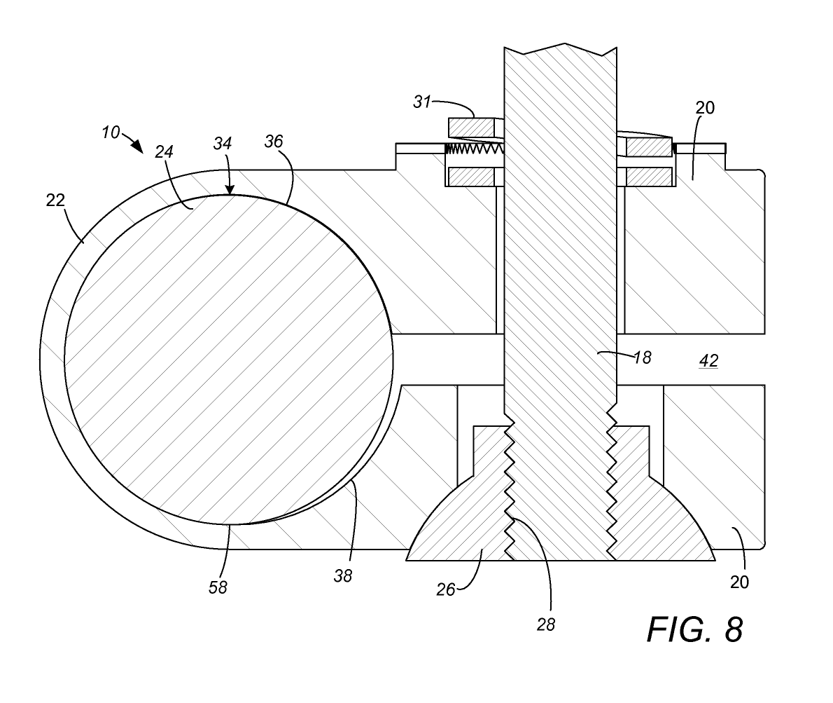

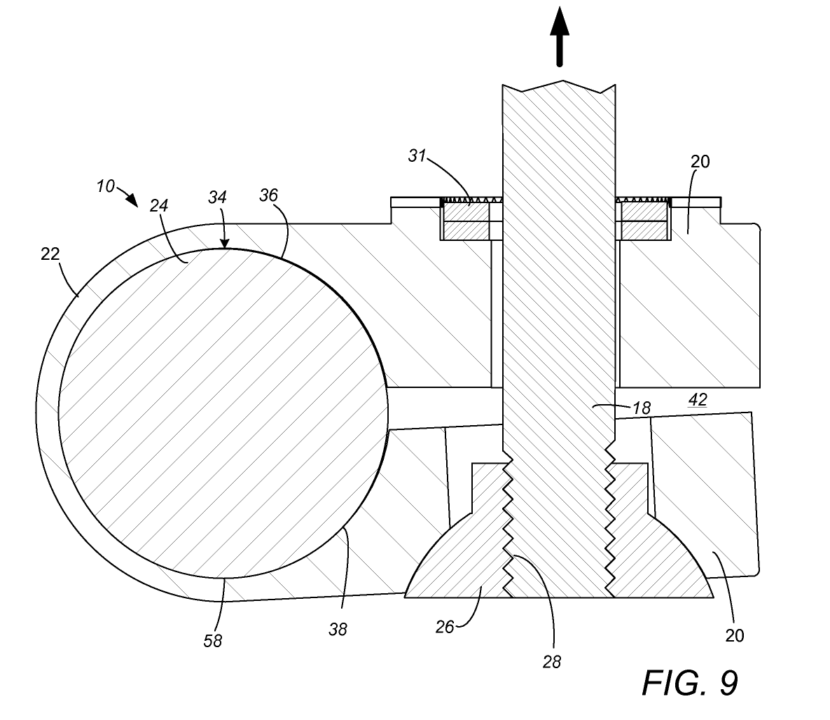

Each clamp 10 is in a generally U-shape including two arms 20 and a base portion 22. The base portion 22 receives cylindrical stock 24 (shown in FIGS. 8 and 9) such as a post or bar of the surgical retraction system as known in the art. While recognizing that the clamp 10 could tighten on any cylindrical component of the retraction system, the cylindrical stock will be hereinafter referred to as a “post” 24 merely for ease of reference. By drawing the two arms 20 of the clamp 10 together, the clamp 10 tightens on the post 24.

The handle 14 with its integrally formed cam 16 and clamp bolt 18 are used to draw the two arms 20 of each clamp 10 together and tighten the clamp 10. A ball nut 26 is attached on a lower threaded end 28 of the clamp bolt 18. The clamp bolt 18 extends through a rotation ring 30 housed within the inside clamp 10b. A compression spring 31 may be positioned between the clamps 10 to provide tension on the loosened clamps 10. A cam pin 32 extends through the cam 16 of the handle 14 and through a mating opening in the clamp bolt 18. When the handle 14 is thrown, the cam 16 moves the cam pin 32 which pulls the clamp bolt 18 relative to the rotation ring 30, shortening the distance between the rotation ring 30 and the ball nut 26. This shortening of the distance between the rotation ring 30 and the ball nut 26 draws the arms 20 of each clamp 10 closer together so each clamp 10 tightens on its post 24.

The various components of the clamp 10 can be formed of any material which can be sterilized and is sufficiently strong for use in a retraction system. The preferred material is 17-4 stainless steel.

As shown in FIGS. 8 and 9, the base 22 of the U-shape is sized to mate with its post 24, such as posts of 1 inch outer diameter. However, in contrast to prior art U-type clamps, the clamp 10 of the present invention has a contact surface 34 which is not a uniform cylindrical profile to match the outer profile of the post 24. Instead, the contact surface 34 is machined to have two distinct profiles 36, 38. Recognizing that the exact shape of the base 22 changes during tightening of the clamp 10, the shapes of the two distinct profiles 36, 38 will be primarily described in their loosened state.

A first portion 36 of the contact surface 34 has a cylindrical profile intended to mate closely with the cylindrical outer profile of the post 24. For instance, for use with 1 inch outer diameter post 24, the first portion 36 is machined to have an inner diameter 40 of 1 inch. This first portion 36 preferably extends around a significant circumferential portion of the contact surface 34. For instance, the contact surface 34 in the preferred embodiment leaves a tightening gap 42 between the two arms 20 of about ⅙ of an inch, and thus the entire contact surface 34 extends around for about 346° of contact with the post 24. The first portion 36 of the contact surface 34 may extend around for a contact angle 44 of 180° or more with the post 24, such as extending around approximately ¾ of the circumference of the post 24. In the preferred embodiment, the first portion 36 extends around for a contact angle 44 of about 263° with the post 24.

A second portion 38 of the contact surface 34 is distinctly different than the first curvature of the first portion 36, such as having a different radius of curvature 46 in the loosened state. The preferred radius of curvature 46 of the second portion 38 is slightly larger than the radius of curvature 40 of the first portion 36, such as about 0.001 to 0.05 inches larger. For example, the second radius of curvature 46 may be about 2% larger than the first radius of curvature 40. When mating with a 0.5 inch radius post 24, for instance, the radius of curvature 46 of the second portion 38 of the contact surface 34 may be about 0.51 inches.

The second portion 38 of the contact surface 34 preferably extends around for a smaller contact angle 48 than the first contact angle 44, such as a contact angle 48 less than 180°, and more preferably a contact angle 48 of no more than 90° with the post 24, thereby making contact during tightening with ¼ or less of the circumference of the post 24. In the preferred embodiment, the second portion 38 extends around for a contact angle 48 of about 83° with the post 24.

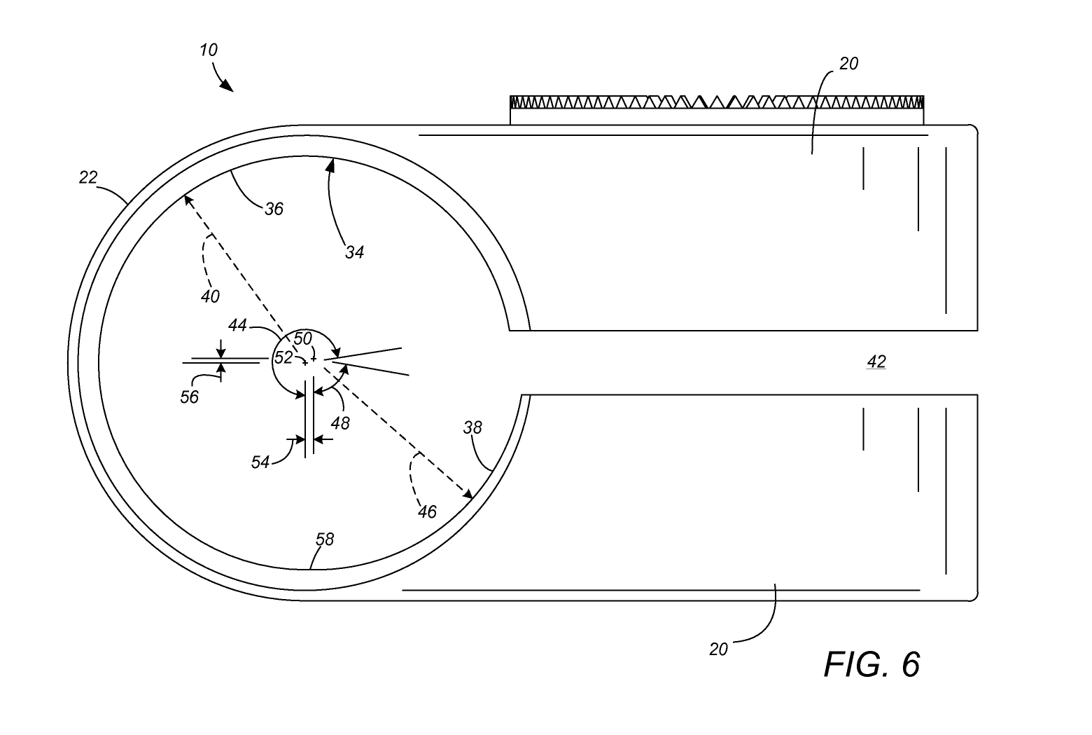

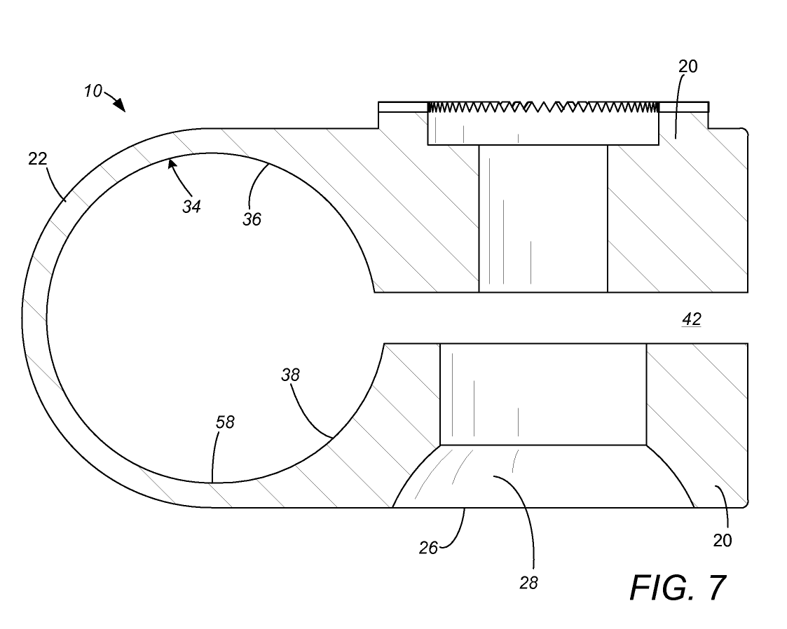

The center 50 of this radius of curvature 46 of the second portion 38 may also be located differently than the center 52 of the radius of curvature 40 of the first portion 36. The axis 50 defined by the second radius of curvature 46 is preferably offset 0.001 to 0.05 inches to the side of the axis 52 defined by the first radius of curvature 40, such that the second portion 38 does not make tight contact with the post 24 in the loosened configuration. For instance, in the preferred embodiment, the axis 50 defined by the second radius of curvature 46 has a 0.02 inch offset 54 to the side of the axis 52 defined by the first radius of curvature 40. To account for the larger radius of curvature 46 of the second portion 38, the two axes 50, 52 also have an offset 56 of a corresponding amount (i.e., the second axis 50 is 0.01 inches vertically higher than the first axis 52 as depicted in FIG. 6).

If there is a side offset 54 between the axes 50, 52 of the two contact portions 36, 38, then it is preferred that the curves be joined without a point of inflection in the contact profile 34 of the clamp 10. For instance, the first portion 36 and the second portion 38 could be simply machined in the clamp 10 as cylinders, but that would leave a small corner or bump in the contact profile 34 with the post 24. Any such corner or bump tends to leave indentations in the post 24 when the clamp is tightened onto the post 24. Accordingly, the preferred embodiment includes a flat 58 which is equal in length to the side offset 54 between the axes 50, 52 of the two contact portions 36, 38 (i.e., a flat 58 which is tangential to both curvatures). This flat 58 could also be replaced with a radius of curvature which is larger than both radii of curvature 40, 46 of the first and second portions 36, 38, tangential with both curvatures, avoiding the indentation problem.

FIG. 9 shows the preferred clamp 10 tightened relative to the post 24. When the two arms 20 are drawn toward each other, the base 22 of the clamp 10 deforms so the entire contact surface 34 including both the first portion 36 and the second portion 38 come into contact with the post 24. In use, a clamp 10 with two contact profiles 36, 38 in accordance with the preferred embodiment has been surprisingly found to more tightly clamp to various posts within tolerances found in the field than prior art clamps.

The physical phenomenon responsible for this better performance is not entirely known. One possible explanation for the tighter grip achieved by the present invention is the possibility of a latent frictional stress between the clamp 10 and the post 24 created during tightening. Rather than have all portions of the contact area equally pressed against the post 24, the clamp 10 of the present invention clearly makes contact with the post 24 along the first portion 36 prior to deforming the second portion 38 into contact with the post 24. This fact, that the second portion 38 of the contact surface 34 does not make contact until a normal force and associated friction is established between the first portion 36 of the contact surface 34 and the post 24, apparently leads to an enhanced frictional gripping between the clamp 10 and the post 24. Another possible explanation for the tighter grip involves a different distribution of normal forces between the post 24 and the clamp 10 during tightening. In any event, the tighter clamping achieved by the present invention occurs with a cam 16 and handle 14 consistently at a location where the handle 14 completes its throw, i.e., within plus or minus 20° of the tightened position shown in FIGS. 1-3 depending upon the specific diameter and any imperfections in the shape of the cylindrical post 24 and upon the amount of tightening force applied.

Although the present invention has been described with reference to preferred embodiments, workers skilled in the art will recognize that changes may be made in form and detail without departing from the spirit and scope of the invention.

1. A U-type surgical clamp comprising:
a first leg of a U-shape;
a second leg of the U-shape; and
a base of the U-shape connecting the first leg to the second leg, such that the U-type surgical clamp can be tightened onto cylindrical stock by drawing the first leg and the second leg closer together, wherein the base defines a contact surface for contacting the cylindrical stock, wherein the contact surface, in its loosened state, comprises:
a first portion having a first curvature; and
a second portion having a second curvature distinctly different from the first curvature.

2. The U-type surgical clamp of claim 1, wherein the first portion is cylindrical, and wherein the second portion is cylindrical.

3. The U-type surgical clamp of claim 2, wherein the first portion has a first radius of curvature, and wherein the second portion has a second radius of curvature which is larger than the first radius of curvature.

4. The U-type surgical clamp of claim 3, wherein the first portion and the second portion are connected with a flat.

5. The U-type surgical clamp of claim 4, wherein the first radius of curvature defines a first central axis, wherein the second radius of curvature defines a second central axis, with the second central axis being offset relative to the first radius of curvature.

6. The U-type surgical clamp of claim 4, wherein the flat has a length of 0.001 to 0.05 inches.

7. The U-type surgical clamp of claim 3, wherein the second radius of curvature is within the range of 0.001 to 0.05 inches larger than the first radius of curvature.

8. The U-type surgical clamp of claim 1, wherein the first portion extends over at least 180° of contact area.

9. The U-type surgical clamp of claim 8, wherein the second portion extends over no more than 90° of contact area.

10. The U-type surgical clamp of claim 1, wherein the first portion has a first radius of curvature, and wherein the second portion has a second radius of curvature which is larger than the first radius of curvature.

11. The U-type surgical clamp of claim 10, wherein the first portion and the second portion are connected with a flat.

12. The U-type surgical clamp of claim 10, wherein the first portion extends over at least 180° of contact area, and wherein the second portion extends over no more than 90° of contact area.

13. The U-type surgical clamp of claim 1, wherein the first portion differs from the second portion in at least one of radius of curvature and axis location.

14. The U-type surgical clamp of claim 1, further comprising a bolt for drawing the first leg and the second leg closer together, a cam coupled to the bolt for moving the bolt, and a cam handle attached to the cam for rotating the cam.

15. A U-type surgical clamp comprising:
a first leg of a U-shape;
a second leg of the U-shape; and
a base of the U-shape connecting the first leg to the second leg, such that the U-type surgical clamp can be tightened onto cylindrical stock by drawing the first leg and the second leg closer together, wherein the base defines a contact surface for contacting the cylindrical stock, wherein the contact surface comprises:
a first portion having a first radius of curvature; and
a second portion having a second radius of curvature which is larger than the first radius of curvature.

16. The U-type surgical clamp of claim 15, wherein the first portion and the second portion are connected with a flat.

17. The U-type surgical clamp of claim 15, wherein the first portion extends over at least 180° of contact area, and wherein the second portion extends over no more than 90° of contact area.

18. The U-type surgical clamp of claim 15, further comprising a bolt for drawing the first leg and the second leg closer together, a cam coupled to the bolt for moving the bolt, and a cam handle attached to the cam for rotating the cam.

19. A surgical clamping system comprising:
a generally cylindrical bar having a bar radius; and
a U-type clamp for clamping onto the generally cylindrical bar, comprising:
a first leg of a U-shape;
a second leg of the U-shape; and
a base of the U-shape connecting the first leg to the second leg, the base being generally sized to mate with the generally cylindrical bar, the base defining a contact surface which comprises:
a first portion having a radius of curvature which matches the bar radius; and
a second portion having a radius of curvature which is larger than the bar radius;
a bolt for drawing the first leg and the second leg closer together;
a cam coupled to the bolt for moving the bolt; and
a cam handle attached to the cam for rotating the cam, the cam handle having a throw to tighten the clamp so as to force the second portion into contact with the generally cylindrical bar.
